(19) United States
(12) Patent Application Publication
(10) Pub. No.: US 2011/0054340 A1
(43) Pub. Date: Mar. 3, 2011

(54) INDICATIVE CHEST SEAL

(75) Inventors: Corey Russ, Fayetteville, NC (US); Christopher Murphy, Vass, NC (US); Brian Farrell, West Des Moines, IA (US); Keith High, West Des Moines, IA (US); Sean Mahoney, Urbandale, IA (US)

(73) Assignee: COMBAT MEDICAL SYSTEMS, LLC., Fayetteville, NC (US)

(21) Appl. No.: 12/852,346

(22) Filed: Aug. 6, 2010

Related U.S. Application Data
(60) Provisional application No. 61/231,888, filed on Aug. 6, 2009.

(51) Int. Cl. A61B 5/08

(52) U.S. Cl. 600/529

(57) ABSTRACT

A wound dressing can include an adhesive dressing with a diaphragm vent that allows the passage of gas out of a patient's body but restricts the flow of gas into the body. A sensor device is positioned so as to be able to sample gas passing through the vent and measure one or more gas characteristics, such as temperature, humidity, and/or pressure. If the data from the sensor device exceeds certain parameters suggestive of a pneumothorax, a processing device can activate an alert system, such as an indicator light, audible alarm, and/or wireless signal, to alert the care provider.

### RELATED APPLICATIONS

The present application claims priority under 35 U.S.C. §119(e) to U.S. Provisional Application No. 61/231,888 filed on Aug. 6, 2009, the disclosure of which is incorporated by reference herein in its entirety.

### BACKGROUND OF THE INVENTION

1. Field of the Invention

This disclosure relates generally to devices for wound treatment and specifically to devices for wound treatment that provide information about a wound.

2. Description of the Related Art

The outer portion of the lung is composed of two tissue layers: the internal visceral pleura that covers the lungs and the external, parietal pleura that is attached to the chest wall. The space between the visceral and parietal pleura is known as the pleural space. Under normal conditions, the visceral and parietal pleura are nearly flush against one another and the pleural space usually contains only a thin layer of pleural fluid. However, if air enters the pleural space, the resulting air pocket can compress the inner portion of the lung leading to a condition known as a pneumothorax, known colloquially as a “collapsed lung.”

Pneumothoracies are generally divided into two types: tension and non-tension. The latter can occur with trauma, various illnesses, or even spontaneously. While non-tension pneumothorax can lead to chest pain and difficulty breathing because the lung is incapable of expanding fully, it is generally of lesser concern than tension pneumothorax, which is a medical emergency. In tension pneumothorax, the air from the lung directly enters the pleural space and becomes trapped therein. The pressure inside the pleural space can increase with every breath, causing the pleural space to expand at the expense of the lung. The result is that the lung is gradually crushed by the rising intrapleural pressure. In some cases, the lung can be pushed into the heart and the other lung, interfering with the functions of these organs. As the blood vessels of the lung are compressed, the vascular pressure increases and puts pressure on the right ventricle of the heart, possibly leading to cardiac failure.

A variety of treatments have been used on chest wounds that can potentially help to relieve intrapleural pressure in the event of a tension pneumothorax. Placing an airtight seal over a chest wound is generally not recommended because it can trap air in the chest cavity, maintain pressure in the intrapleural space, and further exacerbate the tension pneumothorax. Some chest dressings have been developed with a vent so as to allow the release of trapped air.

Given the seriousness of tension pneumothoracies, medical professionals generally need to treat them quickly through the performance of a needle thoracostomy and insertion of a chest tube into the pleural space so as to relieve the intrapleural pressure. It is also sometimes important to treat a tension pneumothorax prior to any attempt to evacuate the patient by air as lowered atmospheric pressure would only exacerbate the problem.

### SUMMARY OF THE INVENTION

Disclosed herein are embodiments of indicative chest seals and discrete components thereof. In some embodiments, a pneumothorax detection system comprises a valve assembly and sensor unit capable of detecting a pneumothorax. Some embodiments of the indicative chest seal can be relatively inexpensive disposable units that can be issued as part of a first aid kit for first responders or medical personnel. Typical users can include paramedics, military personnel trained in first aid or health care providers in a hospital setting. The indicative chest seal can be prepackaged in a sterile wrapper from which it can be removed and easily activated by the user.

The wound dressing can comprise an adhesive dressing with a vent through which gasses escaping from a pneumothorax can pass. A sensor device is positioned so as to be able to sample gasses passing through the vent. The gasses passing through the vent in the adhesive dressing can pass through a diaphragm that allows the passage of gasses into the unit but restricts the flow out of the unit. When sufficient gas pressure is applied to the diaphragm, the diaphragm can open and allow gas to enter a first and then second gas chamber. This second gas chamber can comprise a sensing device capable of measuring one or more gas characteristics, such as temperature, humidity, and/or pressure. The sensing device can be operably coupled to a processing device capable of analyzing data therefrom. If the data from a sensing device exceeds certain parameters suggestive of a pneumothorax (e.g., a rise in temperature, humidity and/or pressure), the processing device can activate an alert system. The alert system can comprise a visual means of alert such as an indicator light, an audible alert system, and/or a wireless alert system that can be detected by a remote receiving device. In some embodiments, the alert system can transmit information, such as the number of times that the sensing devices have recorded data suggestive of a pneumothorax.

In some embodiments, the user can activate the unit by pulling out an activation tab that removes a barrier between the battery and the processing device, thereby powering and activating the unit. Some embodiments can comprise a control switch, enabling the user to deactivate the alert system or toggle among different alert modalities.

### BRIEF DESCRIPTION OF THE DRAWINGS

FIG. 1 is a perspective view of a pneumothorax device.

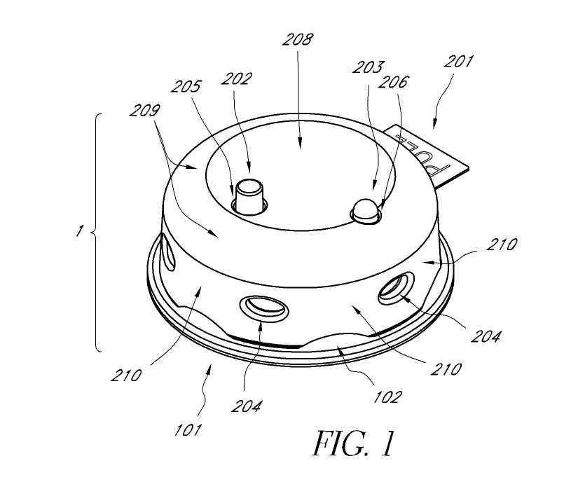

FIG. 2 is a first lateral view of the pneumothorax device.

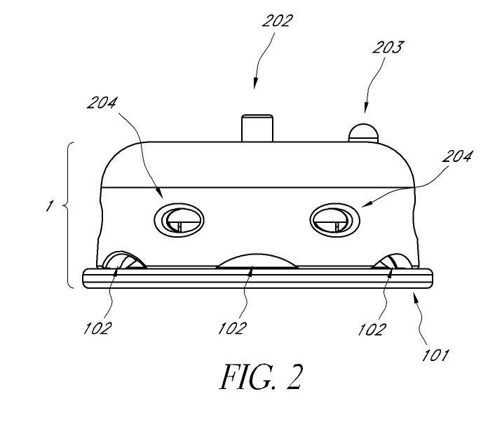

FIG. 3 is a second lateral view of the pneumothorax device.

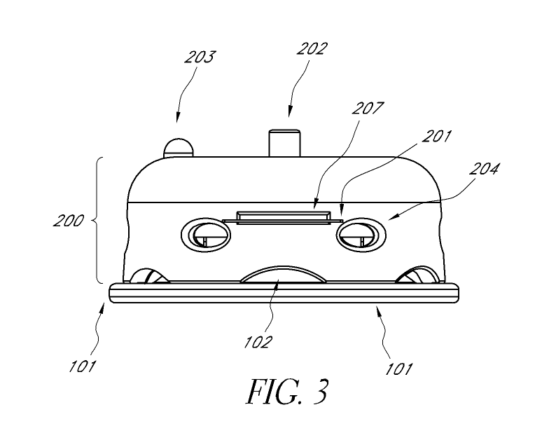

FIG. 4 is a third lateral view of the pneumothorax device.

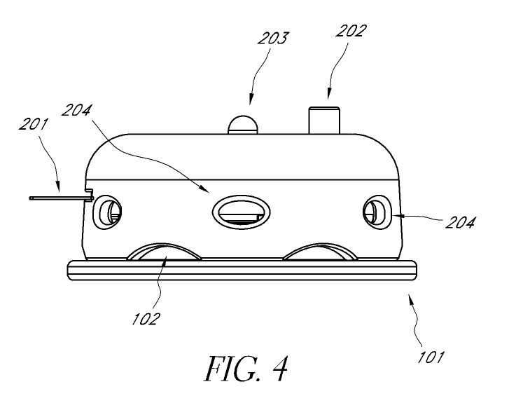

FIG. 5 is a fourth lateral view of the pneumothorax device.

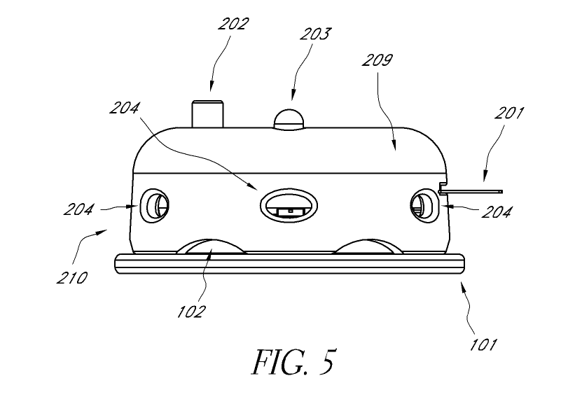

FIG. 6 is a top view of the pneumothorax device.

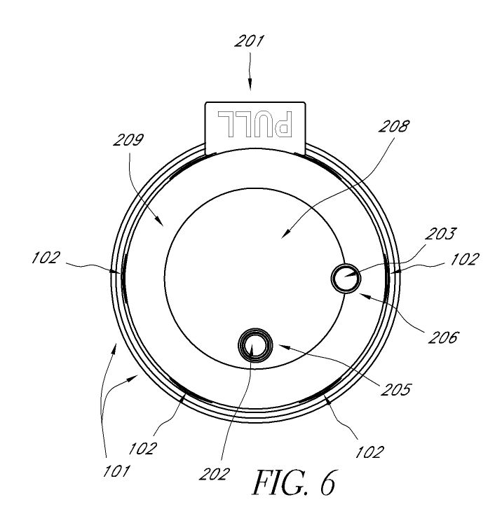

FIG. 7 is a bottom view of the pneumothorax device.

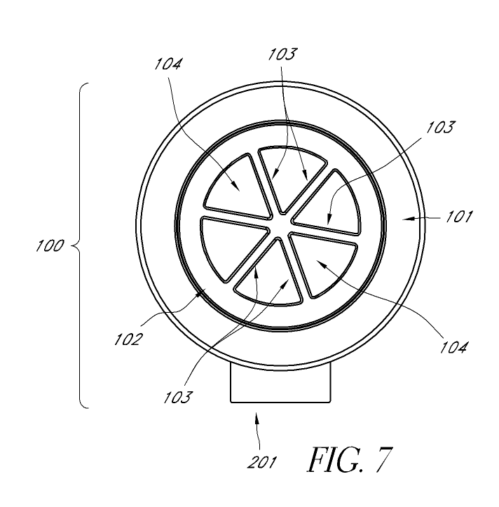

FIG. 8 is a bottom perspective view of the pneumothorax device.

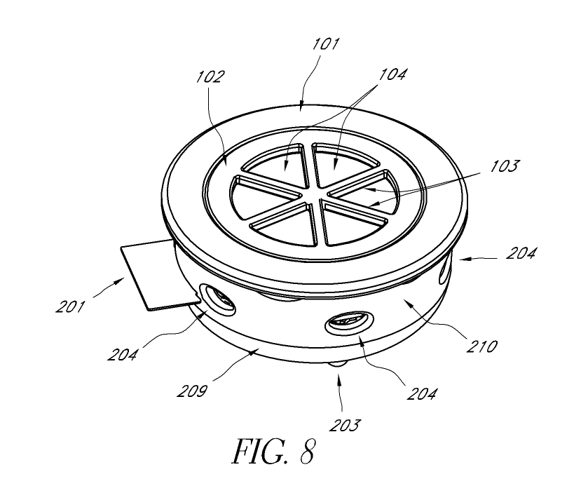

FIG. 9 is a first cross section of the pneumothorax device.

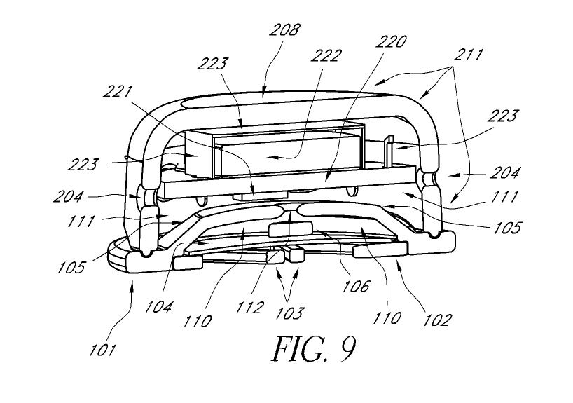

FIG. 10 is a second cross section of the pneumothorax device.

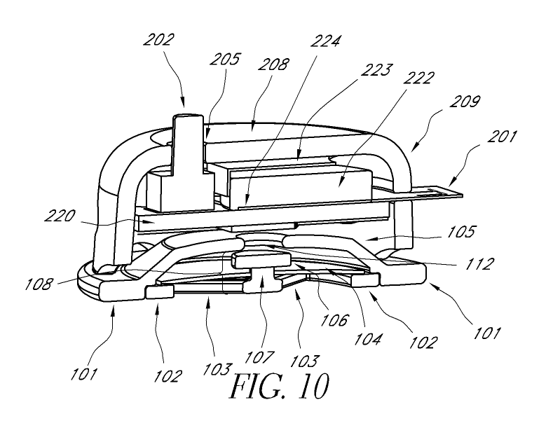

FIG. 11 is a view of an indicative chest seal, including a wound dressing.

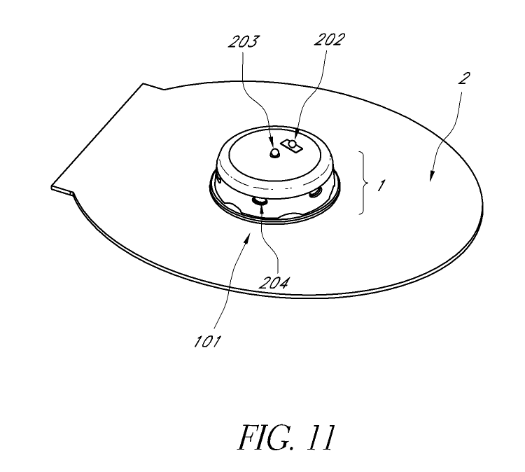

FIG. 12 is a view of the ventilated wound dressing separated from the pneumothorax detection device that is, in turn, separated into its valve assembly and sensor unit components.

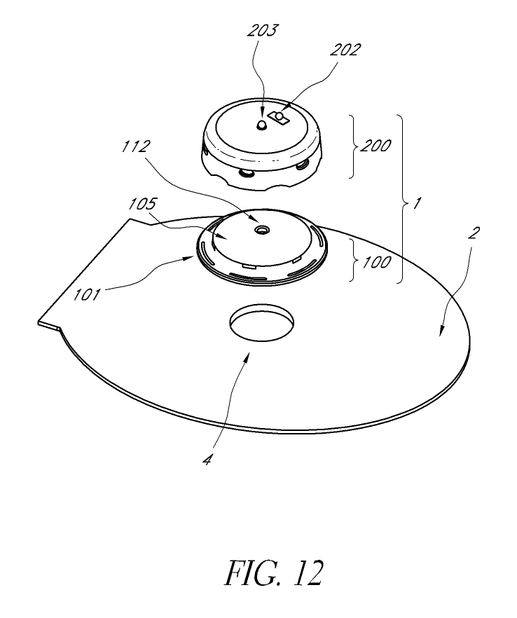

### DETAILED DESCRIPTION OF THE PREFERRED EMBODIMENT

In some embodiments, an indicative chest seal 3 (hereafter ICS 3) can comprise a pneumothorax detection device 1 mounted on a base comprising a ventilated wound dressing 2 (hereafter “wound dressing”). The pneumothorax detection device 1 itself can comprise a valve assembly 100 and a sensor unit 200. In some embodiments, the pneumothorax detection device 1 is a small, lightweight unit, typically a few centimeters in diameter. Many of the structural components of the pneumothorax detection device 1 can be constructed of materials such as plastic, abs plastic, polyvinyl chloride, aluminum or another type of metal.

In some embodiments, the wound dressing 2 comprises a commercially available HALO chest seal. However, a variety of embodiments other than the HALO chest seal are possible. Various embodiments of the wound dressing 2 can have a variety of shapes and sizes appropriate for secure placement over chest wounds. For example, embodiments can be circular, substantially circular, rectangular, or any number of shapes. In some embodiments, the wound dressing 2 can be greater than or equal to about 10 centimeters and/or less than or equal to about 20 centimeters in length and width; however, a variety of sizes are possible as wound dressing 2 component of the ICS 3 can comprise virtually any type of wound dressing material or chest seal.

The wound dressing 2 can be self-adhesive. In some embodiments, there can be a removable backing that the operator can remove to expose the adhesive surface prior to applying to the patient. The wound dressing 2 can be sufficiently adhesive so that it will not become easily dislodged with movement by the patient. In some embodiments, the wound dressing 2 can be sufficiently flexible to help to promote adherence when the patient moves. However, it generally should not be so adherent that the patient's skin will be damaged when the wound dressing 2 is removed. In some embodiments, the wound dressing 2 is not self-adhesive and can be affixed to the patient by taping the dressing to the patient or by applying an adhesive material to the inferior surface of the dressing.

In some embodiments, the wound dressing 2 comprises multiple layers, including (from the inferior surface to the superior surface) an adhesive layer, a bottom permeable layer, a hydrophilic layer, and a second top layer covering the hydrophilic layer. In some embodiments, the hydrophilic layer comprises a hydrogel or similar substance. In some embodiments, the hydrophilic layer can absorb fluids and moisture from the patient, help to keep the wound dry, reduce the risk of infection, and promote healing. In some embodiments, the top and bottom layers are fused together around the edges of dressing so as to contain the hydrogel therein. In some embodiments, the top layer can be impermeable to fluids and gasses.

The wound dressing 2 can comprise a vent 4 in the form of one or more openings at or near the center of the dressing 4. The vent 4 can be circular, substantially circular, or another shape. The bottom layer and the top layer of the wound dressing 2 can be fused around this opening so as to prevent the leakage of the hydrogel layer. The diameter of the vent 4 can vary. In some embodiments, it can range from less than one centimeter to several centimeters. In some embodiments, the vent 4 can be large enough to allow a sufficient amount of gas to pass through the wound to relieve gas pressure and to allow for effective gas sampling, but not so large as to compromise the adhesive properties or structural integrity of the wound dressing 2 or to prevent the attachment of a pneumothorax detection device 1 to the wound dressing 2. In some embodiments, the vent 4 can be surrounded by a collar that can be securely affixed to the wound dressing 2 and that can help to hold the sensor device in position.

In some embodiments, a valve assembly 100 can be positioned on the superior surface of the wound dressing 2 so as to generally or entirely cover the vent 4. The valve assembly 100 can be attached to the collar surrounding the vent 4. In some embodiments, the valve assembly 100 can be affixed directly to the wound dressing 2. In some embodiments, the valve assembly 100 can be affixed to the wound dressing 2 through the use of an adhesive or via mechanical means such as a system of small bolts or clamps.

The valve assembly 100 can comprise a circular or substantially circular outer collar 101. This outer collar 101 can be affixed directly to the wound dressing 2 via an adhesive or other mechanism of attachment. The outer collar 101 can also serve to hold a valve frame 102 in position at the site of the vent 4. In addition, the outer collar 101 can also serve as an attachment point for a sensor unit 200 (described below). In addition to the outer collar 101, the inferior portion of the valve frame 102 can comprise a series of radial projections 103. The gaps between the various radial projections 103 can be sufficiently wide to allow for the passage of gasses. The superior part of the valve assembly 100 can comprise a valve assembly dome 105 with an interchamber vent 112. In the illustrated embodiment, within the valve assembly 100 is a first valve assembly gas chamber 110 wherein gasses can enter through the gaps among the radial projections 103 when the diaphragm 104 is in a raised/open position, and can subsequently exit through the interchamber vent 112 of the valve assembly dome 105.

The valve frame 102 can be positioned within an outer collar 101 and can be held in position thereby. The valve frame 102 can comprise a circular framework that generally or substantially surrounds the vent 4. In some embodiments, the valve frame 102 can comprise a plurality of radial projections 103 extending from the inner surface of the valve frame 102 so as to converge at a position at or approximately above the center of the vent 4. The gaps among these radial projections 103 can allow gasses to enter the valve assembly 100 from the vent 4 of the wound dressing 2. The remainder of vent 4 can be occluded by the inferior surface of the valve frame 102 and/or outer collar 101.

In some embodiments, there can be a valve retainer, such as a T-shaped projection 108, that helps to retain a valve member, such as a diaphragm 104. In the illustrated embodiment, the T-shaped projection 103 rises above the convergence site of the radial projections 103. The T-shaped projection 108 can comprise a shaft 107 and a wider top component 106.

In some embodiments, the diaphragm 104 is a gas impermeable seal that functions as a valve that can be opened by gasses passing in one direction only. In some embodiments, the diaphragm 104 is circular with a small hole in the middle that the vertical shaft of the T-shaped projection 107 can pass through. In some embodiments, the diaphragm 104 is mounted on the shaft of the T-shaped projection 107 so as to limit lateral movement, and the diaphragm 104 can move vertically to some extent along the shaft of the T-shaped projection 107. The movement of the diaphragm 104 can be limited superiorly by the presence of the top component of the T-shaped projection 106 and inferiorly by the valve frame 102 and/or radial projections 103. The diaphragm 104 can be generally round and positioned so that it can form a gas seal above the gaps between the radial projections 103, thereby preventing the passage of gasses therethrough. In some embodiments, the diaphragm 104 can be configured in a concave shape with the outer edges facing down in the direction of the radial projections 103 and valve frame 102. In some embodiments, the outer edge of the diaphragm 104 can be in contact with the valve frame 102 when in the down/closed position thereby forming a seal impermeable to gasses.

The diaphragm 104 can function as a valve allowing the passage of gasses into the first gas chamber 110, while restricting the passage of gasses out of the first gas chamber 110. The biased position of the diaphragm 104 can be in a down/closed position wherein it is substantially flush against the superior surface of the radial projections 103. The edges of the diaphragm 104 can be flush against the valve frame 102 when in the down position in some embodiments, thereby preventing the passage of gasses between the vent 4 and first gas chamber 110. When the diaphragm 104 is in the down/closed position, it can impede gasses within the first gas chamber 110 from pushing it open, as the diaphragm 104 is already generally sealed against the valve frame 102. However, if a sufficient quantity of gas pressure pushes against the diaphragm 104 from below, it can force the diaphragm 104 up along the shaft of the T-shaped projection 107, thereby lifting the diaphragm 104 away from the valve frame 102, and thereby allowing the passage of gasses in the resultant gap between these two components. If gas pressure coming from the vent 4 exceeds that of the first gas chamber 110, the diaphragm 104 can open, and if the gas pressure within the first gas chamber 110 is generally equal to or higher than the gas pressure coming from the vent 4, the diaphragm 104 can remain shut. In some embodiments, the diaphragm 104 can open with small changes in pressure coming through the vent 4. In some embodiments, the diaphragm 104 can be opened by differences in pressure of about 1 mm Hg or less; however, a variety of minimum opening pressures are possible.

In some embodiments, the down/closed bias of the diaphragm 104 can be maintained by gravity alone. In other embodiments, a closure mechanism, such as a small spring or other source of tension between the diaphragm 104 and the top component of the T-shaped projection 106 can keep the diaphragm 104 shut until sufficient air pressure is applied through the vent 4 to open it. In some embodiments, small amounts of pressure, typically about 1 mm Hg or less can move the diaphragm 104 into the raised/open position and allow for the passage of gasses.

In some embodiments, the valve assembly dome 105 covers the superior portion of the valve assembly 100. The valve assembly dome 105 can be affixed to the outer collar 101 and can encase the valve frame 102 and T-shaped projection 108. The interchamber vent 112 can be positioned at or near the center of the valve assembly dome 105 in some embodiments. In some embodiments, the upward sloping roof of the valve assembly dome 105 can help to focus the flow of any gas passing through the diaphragm 104 through the interchamber vent 112 via a venturi effect, thereby facilitating the detection of gasses passing therethrough. The valve assembly dome 105 and outer collar 101 can be airtight; in addition, the diaphragm 104 can restrict the outflow of gasses back into the vent 4 when in the closed position. Thus, gasses entering through vent 4 can be directed through the interchamber vent 112 to escape from the first gas chamber 110 when the diaphragm 104 is in the closed position.

A sensor unit 200 can be positioned superior or functionally near the valve assembly 100. The sensor unit 200 can comprise an outer casing 211 perforated by one or more gas exit vents 204. The outer casing 211 can house a circuit board 220, a sensor array 221, and a processing device. The processing device can be integrated into the circuit board 220 in some embodiments. The sensor unit 200 can comprise a battery 222 or other power source. In some embodiments, the outer casing 211 can comprise one or more access points 205 and 206, wherein one or more indicator lights 203, an alert toggle switch 202 and an activation tab 201 or other components are visible to and/or can be manipulated by an operator.

In some embodiments, the sensor unit 200 assembly and valve assembly 100 can have similar dimensions of length, width, and/or diameter so that they can appear as a single compact unit and thereby minimize the possibility of snagging. The point of contact between the sensor unit 200 assembly and the valve frame 102 can be impermeable to gasses. However, in some embodiments, one or more gas exit vents 204 and 102 can be located at the point of contact between the sensor unit 200 assembly and the valve assembly 100.

In some embodiments, the valve assembly 100 and the sensor unit can be removably attached. In some embodiments, a plurality of projections from the outer collar 101 of the valve assembly 100 can apply pressure on the outer casing 211 of the sensor unit 200 so as to help secure the sensor unit 200 into position. In some embodiments, the sensor unit 200 can be removed from the valve assembly 100. In some embodiments, the sensor unit 200 assembly can be threadably attached to the outer collar 101. In other embodiments, the sensor unit 200 assembly can be removably attached to the outer collar 101 of the valve assembly 100 by a plurality of projections on the outer surface of the valve assembly 100 that can overlap with complementary projections on the surface of the sensor unit 200 assembly and thereby secure the latter into position.

In some embodiments, the sensor unit 200 and valve assembly 100 can be permanently fixed into position. These components can be attached at the site of manufacture or via an adhesive or any other means of attachment such as fusing or soldering the components together. In some embodiments, one or more of the removable attachment structures (e.g., threadable attachment, the use of complimentary projections, etc.) can also be used to further secure the attachment the valve assembly 100 and sensor unit 200.

In some embodiments, the ability to remove the sensor unit 200 from the valve frame 102 can enable a user to change a battery 222 or other power source that can be located on the inferior surface of the sensor unit 200. In some embodiments, the indicative chest seal is disposable as a unit and the battery is not generally replaceable.

The sensor unit 200 can normally be positioned on the valve assembly 100 in an inverted manner. The outer casing 211 can have a top portion 208, a beveled transitional edge 209, and a lateral wall 210. In some embodiments, the beveling can reduce the risk of snagging by providing smooth edges.

Within the sensor unit 200 is a second chamber 111 wherein the sensor array 221 can sample gasses therein. In some embodiments, the lateral surfaces of the second gas chamber 111 are comprised of the lateral wall of the outer casing 210 itself. In some embodiments, the superior surface of second gas chamber 111 comprises the circuit board 220 with the sensor array 221. The inferior surface of the second gas chamber 111 can comprise the valve assembly dome 105. Gas can enter the second gas chamber 111 from the first gas chamber 110 through the interchamber vent 112. Shortly after entering the second gas chamber 111, gas can contact the sensor array 221 located within the second gas chamber 111.

Gasses within the second gas chamber 111 can exit through a plurality of exit vents 204 located around the periphery of the outer casing 211. The exit vents 204 can place the second gas chamber 111 in communication with the outside atmosphere. The gas exit vents 204 can be in a variety of shapes including square, round, or oval. In some embodiments, the gas exit vents 204 have no filtration system and are open to the outside air. However, in other embodiments, the exit vents 204 can have a diaphragm or valve system that can inhibit gasses from entering the second gas chamber 111 through said exit vents 204 and thereby create an erroneous reading.

The sensor unit 200 can comprise a circuit board 220. The circuit board 220 can be affixed to the inner walls of the outer casing 211 and thereby held in position. In some embodiments, the circuit board 220 can be secured in position by one or more braces that help to keep said circuit board 220 into position. In some embodiments, the circuit board 220 comprises a sensor array 221 comprising one or more sensing devices. The circuit board 220 can comprise a processing device. The circuit board 220 can comprise a network of electrical connections that can transmit electrical power from the battery 222 to the other electrical components of the sensor unit 200, can enable the interconnection between the sensor array 221 and the processing device, and/or can enable the processing device to provide power to the alert system.

The circuit board 220 can comprise one or more sensors, such as a sensor array 221 in some embodiments. The sensor array 221 can comprise one or more sensing devices within the sensor array 221 capable of measuring one or more gas characteristics, such as temperature, humidity, gas pressure, and/or gas speed. The sensing devices within the sensor array 221 can be positioned so that they can detect changes in the tested gas parameters within the second gas chamber 111. The sensor array 221 can comprise a sensing device or devices capable of measuring other parameters in some embodiments.

In some embodiments, the circuit board 220 comprises a processing device that can monitor data from an operably coupled sensor device. The processing device can be an integral part of the circuit board 220 and can comprise a microprocessor capable of analyzing data and activating an electrical switching system in response to said data. In some embodiments, the microprocessor can be configured to receive data from the sensor array representing one or more of the foregoing values and can determine from such data when a venting has occurred. When the output from an operably coupled sensing device within the sensor array 221 passes a certain predetermined threshold, or when a combination of outputs in relation pass a certain predetermined threshold, the processing device can activate an operably coupled alert system.

In some embodiments, this predetermined threshold can be a rise in temperature. This can suggest warm air is passing up from an open chest wound, thereby suggesting a tension pneumothorax. In some embodiments, a sensing device within the sensor array 221 capable of measuring humidity can be set to detect a rise in humidity which can suggest that moist air from the body is passing up through the chest wound. This too can suggest a tension pneumothorax.

In some embodiments, a sensing device within the sensor array 221 can directly detect a change in pressure resulting from air passing up from a chest wound into the first and second gas chambers 111. Some embodiments can be configured to detect very small changes in pressure, e.g. about 1 mm Hg. However, a variety of other threshold values are possible. In some embodiments, a change in the measured pressure can activate the alert system. Some embodiments can be configured to monitor two or more measurements simultaneously and/or to produce a combined value from this plurality of measurements to determine when a chest venting has occurred, which can in turn activate an alert system.

In some embodiments, the alarm system of the ICS 3 can include electromechanical components (not shown) for detecting a venting of the chest seal by measuring movement of the diaphragm 104 and/or other valve components.

The sensor array 221 can comprise an operably coupled power source. The power source can comprise a battery 222. In some embodiments, the battery or batteries 222 can comprise a standard button cell or coin cell of the L, S, or C type. However, a variety of configurations are possible. In some embodiments, it may be possible for the user to change the battery 222 by removing the sensor unit 200 from the valve assembly 100 as described above. In such embodiments, the battery 222 can be accessed through the inferior side of the sensor unit 200. In other embodiments, the entire ICS 3 can comprise a disposable unit and the battery 222 permanently fixed into position.

In some embodiments, the battery 222 can provide power to the ICS 3 for an extended period of time, such as at least 18 hours or at least 24 hours. This can generally allow sufficient time for a first responder to provide first aid, seal the chest wound, transport the patient to a medical facility and provide for a thorough evaluation and more definitive medical treatment.

In some embodiments, unnecessary electrical drainage of the battery 222 can be prevented prior to use through the use of an activation tab 201 that physically separates the battery 222 leads from the sensor array 221. This can keep the battery 222 insulated until the user activates the unit thereby prolonging battery life.

In some embodiments, the activation tab 201 can function as the means of activating the sensor unit 200. The activation tab 201 can completely or partially comprise an insulating material that prevents the passage of current. The internal portion of the activation tab 201 can be positioned inside the sensor unit 200 between the battery 222 and the sensor array 221. The activation tab 201 can be kept in position by a breakable portion 223 that can be affixed to a portion of the battery 222 compartment, sensor array 221, or other suitable anchoring site. When sufficient pressure is applied on the activation tab 201 by the user, the breakable portion 223 will break, the activation tab 201 removed from the unit. When the activation tab 201 is removed, any battery leads can be operably coupled to the circuit board 220 and thereby provide power to the sensor array 221, processing device and alert system.

In some embodiments, the activation tab can further comprise printed instructions for the user on how the unit can be activated. In some embodiments, such instructions can be as simple as an instruction to “pull.”

In some embodiments, the battery 222 can be contained within a battery compartment 223 that can secure the battery 222 into position and ensure that the battery 222 can remain in contact with the circuit board 220 and be capable of providing power thereto. The position of the battery 222 can be biased so as to come into contact with the circuit board 220 with the removal of the activation tab 201. The bias can be achieved though the use of a spring or another component positioned on the inside surface the battery compartment 223 that can exert tension on the battery 222 in the direction of the sensor array 221, and thereby keep the battery 222 in functional contact with the circuit board 220 and its operably coupled components when the activation tab 201 has been removed. In some embodiments, the detection system of the ICS 3 cannot be turned off once initially activated to avoid accidental deactivation.

In some embodiments, there can be an alert system comprising a visible, auditory, wireless or other means of alerting the user that the system is active and functioning. This can comprise a transient illumination of an indicator light 203, a brief activation of the auditory alarm, or other means of alerting the user.

The sensor array 221 can be operably coupled to an alert system. The alert system can comprise an indicator light 203, an auditory alarm, a wireless adaptor capable of transmitting the alert to a remote location, or other means of notifying the user that the temperature, humidity, or pressure has exceeded a certain threshold suggestive of possible venting from a pneumothorax. The ICS 3 can also include a data display (not shown), such as an LCD screen for displaying information about the wound, such as the number of ventings, the timing of ventings, the frequency of ventings, and/or the pressure within the wound cavity, etc.

In some embodiments, the alert system can comprise an indicator light 203 visible on the outer surface of the outer casing 211. The indicator light 203 can be operably coupled to the battery 222 and processing device. In some embodiments, the indicator light 203 can pass through the outer casing 211 of the sensor unit 200 through an access point 206 in the outer casing 211. When the alert system is activated, the processing device can activate the indicator light 203.

The indicator light 203 can be powered by the same battery 222 that powers the processing device. In some embodiments, the indicator light 203 can glow steadily once the alert system is activated in one color, such as green, and then transition to a second color, such as red, upon the occurrence of a venting of the chest seal. In some embodiments, the indicator light 203 can flash when the alert system is activated. For example, the indicator light 203 can be set to flash in a certain sequence depending on the data received by the processing device. The indicator can flash once for each venting occurrence since activation. For example, if one venting from a possible pneumothorax is detected, the indicator light 203 can be set to flash once with a pause between flashes. If two ventings are detected, it can be set to flash twice with a pause between pairs of flashes, etc. Multiple venting events can trigger an appropriate number of flashes so as to alert the user of the likelihood and severity of a possible pneumothorax. A variety of such standardized sequences are possible to alert the user to the detection of venting events and the quantity thereof. In some embodiments, the indicator light 203 can comprise an LED. In other embodiments, the alert system can comprise a light of a type other than an LED.

In some embodiments, a venting occurrence can be demonstrated by a change of position of a physical component. For example, in some embodiments, an indicator can be exposed to view from within the ICS 3 to show a venting. In some embodiments, the indicator can be a tab or post with a color that contrasts with the coloring and/or shape of one or more other surfaces of the ICS 3, thereby rendering the indicator readily viewable from a distance. In some embodiments, the indicator can “pop up” or a component initially blocking the indicator can be removed to communicate that a venting has occurred.

In some embodiments, the alert system can comprise an audible alarm system capable of generating a sound that can be heard by an operator. This system can comprise a small speaker operably coupled to the processing device. This speaker can be located near the circuit board 200 or can be positioned at an access point in the outer casing 206. The audible alarm system can be set to deliver a standardized sound such a beep or whistle when the sensor array 221 detects a venting event suggestive of a possible pneumothorax. In some embodiments, the alarm system can beep once, pause, and then beep again in response to a single detected venting event. If two venting events are detected, the alarm system can beep twice, pause, and then beep twice, etc. A variety of such codes are possible to alert the user to the suspected number of venting events.

In some embodiments, the alert system can comprise a wireless transmitter capable of remotely alerting the user to a suspected pneumothorax. This can comprise an infrared or radio transmitter. In such embodiments, the alert system can transmit data via a receivable signal. The signal can be received and processed via a device capable of receiving such signals. A variety of embodiments of such devices are possible including small, handheld monitoring devices that can be carried by first responders or medical personnel. Large fixed monitoring devices in a hospital setting can also be configured to monitor and detect such signals from the alert system.

In some embodiments, the sensor unit 200 can further comprise an alert toggle switch 202 (hereafter switch) that can enable the user to change the output of the alert system. The switch 202 can pass through an access point 205 in the outer casing 211 enabling the operator to access the superior portion of the switch 202. The inferior portion of the switch 202 can interface with the circuit board 220 and can interact with the alert system or the processing device.

The switch 202 can be used by an operator in situations wherein there are serious disadvantages to an alert system comprising a flashing indicator light 203. This can be the case in a combat situation wherein a flashing indicator light can be a target for enemy fire. This switch 202 can comprise a toggle switch, button, touchpad, or other control surface. Said switch 202 is operably coupled to the processing device. In some embodiments, said switch 202 can direct the processing system to disable the alert system entirely. In some embodiments, the same switch 202 can be used to reactivate the alert system. In many such embodiments, the physical position of the switch 202 (e.g., depressed or not depressed) can demonstrate to the user whether the alert system is activated or not. In some embodiments, the switch 202 can deactivate an auditory alert system. In other embodiments, the switch 202 can toggle among some combination of a visual alert and/or auditory alert and no alert.

In some embodiments, when the alert system is deactivated by the switch 202, the ICS 3 can continue to monitor and record venting events. In such embodiments, when the alert system is reactivated, the alert system can notify the user of venting events that took place during all or a portion of the period during which the alert system was deactivated. For example, if no venting events were detected prior to the deactivation of the alert system, and then two venting events are detected while the alert system is deactivated, upon the reactivation of the alert system, the indicator light 203 can flash in a pattern informing the user that two venting events had been detected.

Some embodiments of the ICS 3 can further comprise a timing device or clock. Said timing device can be an integral part of the processing device. In some embodiments, the timing device can be activated with the activation of the ICS 3. The timing device can be configured to record the time when a given venting event is detected. For example, in some embodiments, the timing device can record how long after the installation of the ICS 3 on the patient the venting occurred and/or the timing device can record the time of day when the venting occurred. This data can then be relayed to the alert system and made available in some form to the user. In embodiments wherein the alert system is operably coupled to an external monitoring device, data regarding the timing of venting events can be displayed. For example, a user may be able to see that venting events took place 15 minutes, 19 minutes, and 20 minutes after the ICS 3 was activated. In some embodiments, the external monitoring device can determine how long the ICS 3 has been activated and determine the when such events occurred with reference to the actual time (e.g., a venting event detected at 6:19 PM) or in reference to how much time has passed since the venting event was detected (e.g., 7 minutes ago). The user or the microprocessor can then determine when venting events have occurred, how much time has elapsed since a detected venting event, and any temporal patterns among venting events (e.g., increasing or decreasing in frequency).

Some embodiments of the ICS 3 can be packaged in sterile packaging and ready for use by the user. In such embodiments, when used, the user can remove the ICS 3 from the package, remove the adhesive backing of the wound dressing 2, apply the ICS 3 over the chest wound so that the vent 4 is positioned over the chest wound, and remove the activation tab 201. In some embodiments, the test signal can then inform the user that the system is active.

In some embodiments, as illustrated, the ICS 3 can continue to function as a mechanical chest vent even if the indicative features are not activated, do not function, are not provided, or run out of electrical power. In this way, the patient's chest can continue to vent as a therapeutic treatment for pneumothorax even if the ICS 3 is not being used as a diagnostic tool.

Some embodiments of the ICS 3 can further comprise a pressure sensor that can monitor pressure rising in the wound. In some embodiments, this pressure sensor is positioned on the underside of the ICS 3, and in some embodiments, this pressure sensor is configured to be positioned inside of the wound. For example, the pressure sensor can be tethered to the ICS 3 by way of a wire or other means to secure it to the ICS 3 and to communicate information to the ICS 3. Some embodiments of the pressure sensor can detect very small changes in pressure that are still too small to be detected as a full scale venting event that triggers the device, but can still be suggestive of a pneumothorax or the early development thereof. This additional pressure sensor can be operably coupled to the alert system so as to inform the user of a rise in pressure. In some embodiments, the ICS 3 can provide the user with numerical data showing the amount of pressure inside of the wound. In embodiments using a visible alert system such as an indicator light 203, the said indicator light 203 can glow in one color, such as yellow, when a rise in pressure is detected, and the indicator light 203 can glow in a different color, such as red, when a full venting has occurred. Embodiments utilizing other indicator systems (i.e., audible or remote) can likewise have specific alerts that can inform the user of a buildup of pressure at the wound.

This application provides various examples for purposes of illustration, but the inventions should not be limited to the embodiments illustrated or described. In addition, any features or embodiments described above can be combined with any other features or embodiments disclosed herein or known to those of skill in the art.

The following is claimed:

1. An indicative chest seal for venting a pneumothorax, the indicative chest seal comprising:
a wound dressing comprising a vent;
a valve disposed over the vent that allows gas to exit out the vent and through the valve, but substantially obstructs gas from entering the vent through the valve;
a sensor unit disposed on the chest dressing with an inlet fluidly connected to the valve and an outlet fluidly connected to the surrounding environment, the sensor unit comprising:
a sensing device capable of measuring at least one characteristic of the gas exiting the vent;
a processing device configured to analyze data from the sensing device;
an alert system that is activated when the data exceeds at least one parameter.

2. The indicative chest seal of claim 1, wherein the wound dressing further comprises an adhesive layer configured to adhere to a patient's derma.

3. The indicative chest seal of claim 1, further comprising a battery adjacent the processing device and a barrier between the battery and the processing device, wherein the barrier is removed to activate the sensor unit.

4. The indicative chest seal of claim 1, wherein the sensing device measures one or more of the temperature, humidity, pressure or velocity of the gas exiting the vent.

5. The indicative chest seal of claim 1, wherein the valve comprises a diaphragm that opens when gas exits the vent and closes to substantially obstruct gas from entering the vent.

6. The indicative chest seal of claim 5, wherein the sensing device comprises a device that measures a movement of the diaphragm.

7. The indicative chest seal of claim 1, wherein the alert system comprises an indicator light, audible alarm and/or mechanical indicator.

8. The indicative chest seal of claim 1, wherein the alert system comprises a wireless signal that is sent to a remote receiving device.

9. The indicative chest seal of claim 1, wherein the sensor unit monitors the number of times that the alert system is activated.

10. The indicative chest seal of claim 1, wherein the sensor unit further comprises a control switch.

11. The indicative chest seal of claim 10, wherein the control switch deactivates the alert system or toggles between different alert modalities.

12. The indicative chest seal of claim 1, wherein the sensor unit further comprises a timing device to measure the time between venting events.

13. The indicative chest seal of claim 1, wherein the sensor unit comprises a data display for displaying information about the wound.

14. The indicative chest seal of claim 13, wherein the data display shows one or more of the number of ventings, the time of ventings, the frequency of ventings, or the pressure within the wound.

15. An indicative chest seal comprising:
a wound dressing comprising a vent;
a one-way valve disposed over the vent;
a portable sensor unit with an inlet fluidly connected to the valve and an outlet fluidly connected to the surrounding environment, the sensor unit comprising:
a sensing device capable of measuring at least one characteristic of gas;
an alert system that is activated when the characteristic exceeds a parameter.

16. A method of treating pneumothorax in a patient, the method comprising the steps of:
removing an indicative chest seal from a protective package, the indicative chest seal comprising a wound dressing having a vent, a one-way valve disposed over the vent, and a sensor unit comprising a sensing device capable of measuring at least one characteristic of gas and an alert system that is activated when the characteristic exceeds a parameter;
positioning the indicative chest seal over a wound, wherein a vent of the indicative chest seal is directly over the wound;
monitoring the alert system for indications about the condition of the patient.
